(19) United States
(12) Patent Application Publication
(10) Pub. No.: US 2009/0062868 A1
(43) Pub. Date: Mar. 5, 2009

(54) PEDICLE SCREW

(75) Inventor: Simon Casutt, Gossau (CH)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER GMBH, Winterthur (CH)

(21) Appl. No.: 11/910,004

(22) PCT Filed: Apr. 4, 2006
(86) PCT No.: PCT/EP2006/003059

§ 371 Date: Aug. 5, 2008

(30) Foreign Application Priority Data
Apr. 4, 2005 (EP) 05007292.5

(51) Int. Cl. A61B 17/04

(52) U.S. Cl. 606/316; 606/301; 606/308; 606/315

(57) ABSTRACT

The invention relates to a pedicle screw comprising a head (11) for coupling to an elastic intervertebral stabilizing or supporting system and comprising a shank, which is to be anchored inside a vertebra and which, when implanted, extends through the pedicle and into the vertebral body. The shank comprises, in its longitudinal extension measured from the head: an upper shank area (19) adjacent to the head and a stress-relief area (17), which is joined to the upper shank area, the stress-relive area having a flexural strength less than that of the upper shank area, and the stress-relief area is placed in the area of the shank provided for being placed inside the bone.

The invention relates to a pedicle screw comprising a head for coupling to an elastic intervertebral stabilization or support system and a shaft which serves for the anchorage in a vertebra and extends through the pedicle into the vertebral body in the implanted state. The invention moreover relates to an intervertebral stabilization system comprising a plurality of pedicle screws.

The pedicle screws will also be termed “fastening elements” in the following.

Pedicle screws of this kind and intervertebral stabilization and support systems are known, for example, from US 2005/0154390, U.S. Pat. No. 5,492,442, WO2005/065374, WO03/032862, EP 0 669 109, EP 0 672 388, U.S. Pat. No. 4,950,269 or EP 528 706. Screws of this type have an eye which is closed or open at one side as the head, with the latter shape having the shape of a tulip or of a tuning fork in specific embodiments and with the eye being provided to accept the intervertebral stabilization or support elements and to fix them by suitable measures.

A pedicle into which a screw should be introduced can be considered, in a simplified mechanical model, as a bar fastened to the vertebral body which can bend significantly under strain. The stiffness profile in the direction of the longitudinal axis of the pedicle is not constant. The stiffness is rather considerably increased in the region of the transition between the pedicle and the vertebral body in comparison with the adjacent regions. In this connection, tests have shown that with implanted rigid pedicles such as are conventionally used, the pedicle, including the screw, deflects when a caudal-cranial force engages at the screw head. Due to the much higher bending stiffness of the screw, a pivot point also called a toggle point is formed around which the pedicle screw tilts under strain. This pivot point is generally approximately disposed more or less in the region of the transition between pedicle and vertebral body. The tilting of the loaded pedicle screw results in an excessive load of the spongiosa of the vertebral body by the free end region and in particular by the tip of the pedicle screw. Heavy strain on the so-called bone interface is hereby produced which, under unfavorable circumstances, for example, with advancing osteoporosis, can result in screw loosening phenomena in the long term. In addition, high strains are generated inside the shaft, above all slightly to anterior of the pivot point.

Pedicle screws are used in conjunction with both rigid and resilient intervertebral stabilization systems or fixation systems (stiffening or support systems). In systems which provide a non angle-stable connection or coupling between the intervertebral support elements, for example between bars or ties arranged at a pedicle screw fastened to a caudal vertebra and at a pedicle screw fastened to a cranial vertebra, on the one hand, and the pedicle screws, on the other hand, and the pedicle screws, on the other hand, such as elastic, dynamically stabilizing systems, substantially no torques are introduced into the screw head or absorbed there, but practically only pure tractive/compressive forces. This has the consequence that the pedicle can be deflected. The stiff screw therefore undergoes a type of tilt strain which cannot be supported by the pedicles due to the comparatively low bending stiffness of the pedicles and therefore introduces high forces into the spongiosa of the intervertebral body in the region of the tip of the screw at the so-called bone interface.

This phenomenon occurs in another respect to a much lower degree with stiffening systems which include an angle-invariant coupling than with systems having angle-variant systems. Due to the angle invariability, a tilt strain of the screw is made impossible, i.e. the torque which induces the force at the screw tip via the lever arm from the toggle point to the screw tip in an angle-variant system is compensated directly in the screw head in an angle-invariant system, with the pedicle screw set forth and claimed in the following also easily being suitable for these angle-invariant systems.

A pedicle screw is known from US 2005/154390 which has a zone elastic in flexion in a region more or less directly adjacent to the head and disposed outside the head after implanting.

A pedicle screw of the initially named kind should be set forth which, in addition to other advantageous properties, is also able to avoid a loosening of the screws and simultaneously to ensure a suitable support of torques which arise due to the engagement of forces at the screw head.

The pedicle screw set forth in claim 1 can satisfy this demand in addition to a plurality of further advantageous properties.

The pedicle screw is inter alia suitable for use in intervertebral stiffening or support systems which provide a non angle-stable connection between the intervertebral support elements, for example between bars or ties arranged at a pedicle screw fastened to a caudal vertebra and at a pedicle screw fastened to a cranial vertebra and the pedicle screws. A system of this type is likewise set forth and claimed. The screw set forth here can naturally also develop extremely advantageous effects in other support or stiffening systems or other applications.

With the pedicle screw set forth here, the shaft is provided with a relief zone which has a reduced bending stiffness with respect to an upper shaft region adjacent in the direction of the head, with the relief zone being disposed in the region of the shaft provided for the arrangement in the bone.

The shaft of the fastening element (pedicle screw) is provided with a relief zone which can also be called a flexible transition zone. This represents a turning away from conventional pedicle screws which are made rigid over their total length and have a high bending stiffness in comparison with the bone material of the vertebrae.

Due to the relief zone, the shaft of the fastening element is given a bending stiffness profile whose extent can be set generally as desired by the design of the relief zone and can in particular be matched to the characteristic properties of the vertebra. It has been found that the transfer of bending torques, which are caused by forces engaging at the head of the fastening element, can be limited by the relief zone in the free end region of the fastening element. The shaft of the fastening element can bend under load due to the relief zone, so to say in harmony with the bone structure into which the fastening element is implanted. High strains on the bone material and high stresses in the shaft are herewith avoided. The matching of the bending stiffness of the pedicle screw to the biological circumstances thus provides a further improved security for the patient.

In the intervertebral stabilization system furthermore set forth and claimed, a plurality of pedicle screws, which can be anchored to the vertebrae, and a connection device for the connection of at least two pedicle screws anchored to adjacent vertebrae are provided to form an elastic stiffening or support system.

Conventional pedicle screws frequently have a conical shape and so, strictly speaking, no constant bending stiffness over the shaft length. Such a “bending stiffness profile”, however, actually has no relief zone in the sense set forth here. The pedicle screw set forth here is rather based on the idea of reducing the bending stiffness of the shaft directly in a specific axial region.

Pedicle screws are usually implanted in the region of the lumbar vertebrae L1 to L5. Although these vertebrae are not identical either with the same patient or with different patients, all vertebrae which can be considered for the implanting of pedicle screws coincide with respect to specific characteristics, as already explained above. This circumstance, which will be looked at in more detail in the following, can be utilized in the pedicle screw set forth here in that the bending stiffness profile of the shaft is directly matched to the bone structure formed by the pedicle and the vertebral body. The pedicle screw set forth here is, however, not restricted to the named lumbar vertebra. A use for the thoracic vertebra is also in particular generally possible.

Further embodiments are also set forth in the dependent claims, in the description as well as in the drawing, with the features of these embodiments being able to be combined with one another in any desired manner per se.

In an embodiment, the bending stiffness of the relief zone is substantially matched to that of a pedicle or is slightly above it. The bending stiffness, for example, amounts to 1.5 to 4 times, 1.5 to 3 times, 2 to 4 times or 2 to 3 times the bending stiffness of the pedicles, with this value having to be selected in dependence on the total geometry of the screw such that a surface pressure on the spongiosa tissue is in particular adopted in the region of the screw tip which is below the maximum permitted surface pressure, but permits a utilization of the permitted strain which is as optimum as possible while taking account of a safety factor.

The head can be rigidly coupled to the upper shaft region at least in the bending region of the shaft and can in particular be in one part. In the case of an embodiment not in one part, it is possible, but not necessary, for the coupling admittedly to be fixed with respect to a bend, but for a torsion to be possible, in contrast.

The gradient of the bending stiffness at the transition from the upper shaft region to the relief zone can be larger, in particular significantly larger, in magnitude than a bending stiffness gradient occurring in the upper shaft region.

At the transition from the upper shaft region to the relief zone, the magnitude of the gradient of the bending stiffness can be at least twice as large, in particular at least 5 times as large, and furthermore in particular at least 10 times as large at least in a region of the shaft than in the upper shaft region.

The bending stiffness can reduce substantially abruptly at the transition.

The bending stiffness in the relief zone can be lower in at least one bending plane with respect to the bending stiffness in the upper shaft region by at least 30%, in particular by at least 50%, in a further embodiment by at least 60%, and in a still further embodiment by at least 80%.

The upper shaft region can be dimensioned such that the relief zone is disposed in the region provided for the arrangement of the spongiosa, whereas at least the region provided for the arrangement in the cortex is formed by the upper shaft region. The upper shaft region thus serves for the support at the fixed bone material.

The length of the upper shaft region can amount to 5 mm as a minimum, in particular to 8 mm as minimum, as well as to 15 mm as a maximum and in particular to 12 mm as a maximum.

As already indicated in the above, the bending stiffness or the maximum permitted surface pressure in the vertebra has an extent which basically has the same characteristic for all vertebrae with respect to an axis which extends through the pedicle and the vertebral body and which coincides with the central axis of the fastening element in the implanted state.

This circumstance be utilized in that an axial bending stiffness profile of the shaft is approximated at least to the qualitative extent of the bending stiffness of the pedicle and corresponds to it in the ideal case. It can hereby be achieved that the transverse force exerted by the pedicle screw and extending perpendicular to its longitudinal extent is at least approximately constant along its longitudinal extent.

The relief zone can be disposed between the upper shaft region and a lower shaft region which both have a higher bending stiffness than the relief zone.

At the transition from the relief zone to the lower shaft region, the magnitude of the gradient of the bending stiffness, at least in a region of the shaft, can be at least twice as large, in particular at least 5 times as large and furthermore in particular at least 10 times as large as in the lower shaft region.

With this design, the lower shaft region which is disposed behind the relief zone—considered from the head—therefore also has greater bending stiffness than the relief zone. This design of the fastening element is, however, not necessary. It is basically rather also possible to provide a relief zone extending up to the free end of the fastening element. With a corresponding design, in particular of the tip of the fastening element, a “guide effect” in the pedicle can hereby be utilized. The fastening element is so-to-say “automatically” correctively deflected by the relatively hard cortical outer layer on the introduction. with a correspondingly formed pedicle screw, it would thus be possible so-to-say to screw “around the corner”.

The change in bending stiffness from the relief zone to the lower shaft region can substantially take place abruptly.

The profile of the bending stiffness development between the upper shaft region and the lower shaft region is made substantially in the form of a pot, a trough or a tub.

The position and the axial length of the position of the relief zone in the shaft are matched to the position of the transition zone between the pedicle and the vertebral body in the vertebra for which the fastening element is designed. Since the bone structure of vertebrae is sufficiently known, it is substantially precisely certain at which position along the implanted shaft the mentioned transition zone and the toggle point already mentioned above lie. This at least applies in each case with respect to a specific fitting technique selected by the surgeon. It must, for example, be taken into account that comparatively long pedicle screws are used e.g. for a so-called bicortical anchorage. Against this background, with the pedicle screw set forth here, the relief zone can be disposed in a central region of the longitudinal extent of the shaft, in particular substantially in the two central quarters of the shaft or in the central third of the shaft.

The relief zone can take up a significant axial length of the shaft. If the shaft is provided with a thread, the relief zone can extend over a plurality of thread turns in the longitudinal direction.

The upper shaft region and the relief zone can be dimensioned such that, in the implanted state, the relief zone is in the region of the transition between the pedicle and the vertebral body and in particular extends axially beyond the transition region on both sides, with a section of the relief zone disposed behind the transition viewed from the head having a larger axial length in an embodiment than a section of the relief zone disposed in front of the transition.

The shaft can be provided with a thread, with the thread being interrupted by the relief zone.

The shaft can be made hollow at least regionally and can in particular be provided with a central longitudinal bore.

The longitudinal bore can be uninterrupted, with this, however, not being absolutely necessary. An uninterrupted bore inter alia has the advantage that the fastening element can be guided, for example by means of a Kirschner wire, during the implanting.

A hollow design of the shaft is, however, not compulsory. The shaft can also be made in solid form, with a solid design of the shaft also being able to be provided in the relief zone.

The shaft can be provided with a cross-section attenuation at least in the relief zone in comparison with the upper shaft region and in particular also with a lower shaft region.

The reduced bending stiffness in the relief zone of the shaft can be realized in that the shaft is provided with an attenuation by material removal in the relief zone. This material removal can take place such that the surface torque of the shaft is reduced with a simultaneously ideal utilization of the material forming the shaft.

There are a plurality of possibilities which can be realized in practice for the weakening by material removal. Examples will be sketched briefly in the following. Corresponding specific aspects will be looked at in more detail in connection with the description of the drawings.

The relief zone can be formed by an elongated shaft region having a reduced cross-sectional surface in comparison with the upper shaft region and in particular also with a lower shaft region.

The shaft can be made as a helix at least in the relief zone.

The shaft can be provided with a groove-like or slot-like recess, at least in the relief zone, which in particular extends in a helix shape. The peripheral recess can be oriented in the same sense or in the opposite sense with respect to a thread formed at the shaft.

If the shaft is hollow at least regionally or provided with a central longitudinal bore, provision can be made for the wall of the shaft to be broken open in the shaft regions made hollow.

If it is a question of a shaft provided with a thread, the wall can be interrupted in the thread valley. A design is, however, also possible in which the wall is interrupted at the thread peak.

An embodiment of the shaft of this type is also suitable for those fastening elements which are not screwed into the vertebra, but are rather hammered in, since the hammering impulses can be transmitted without problem in an axial direction thanks to the low slot width.

The fastening element set forth here does not necessarily have to be a case of screws in the conventional senses, but they can also be fastening elements implantable by being hammered in. Nevertheless, the shaft of a hammered in element of this type can be provided with a thread start which does not hinder the hammering in, but facilitates or makes possible an explanting by unscrewing.

The wall of the shaft can have two helically extending slot-like interruptions at least in the relief zone.

The shaft can form a double helix at least in the region of the relief zone.

The pitch of the helix can amount to at least 5 mm, in particular at least 10 mm, in the region of the relief zone.

The pitch of the helix can vary over the longitudinal extent of the shaft, whereby the bending stiffness of the shaft varies over the longitudinal extent.

The manufacture of a relief zone with uninterrupted, also helical or double-helical peripheral grooves or slots can take place, for example, by a wire erosion process known from the prior art. Wire erosion processes have been described, for example, in DE 101 96 821 T5. In the application described here, a transverse bore is introduced into the shaft through which the wire used for the erosion process is guided. Either the screw or the wire is advanced in the axial direction of the screw during the erosion process. The screw is rotated around its longitudinal axis during the advance movement in an embodiment of the method. A helical slot is thereby formed or two helically extending slots are simultaneously produced. A straight line is produced without the rotation of the slot. The machining in particular takes place toward the clamping position of the screw or, with clamping on both sides, toward the clamping position via which the advance force and/or rotational force is applied. This ensures a good force transmission during the machining process. In a variant of the manufacturing process, the head is clamped or driven at the head and the erosion process takes place toward the head.

The shaft can be provided with transverse bores extending perpendicular or obliquely to the shaft axis at least in the relief zone.

Slots whose width is smaller than the diameter of the transverse bores can be arranged leading from the outer wall of the shaft toward the transverse bores.

The shaft can be provided with a plurality of slot-like, groove-like or notch-like recesses arranged sequentially in the axial direction and in particular offset, with the depth of the recesses in each case being larger than half the diameter of the shaft measured in the region of the recess in an embodiment.

It is not necessary for the bending stiffness profile of the shaft to be identical in all planes containing the central axis of the shaft, i.e. a rotational symmetry of the bending stiffness—with respect to the central axis of the shaft—is not absolutely necessary. Consequently, the bending stiffness profile of the shaft can be rotationally asymmetrical with respect to its longitudinal axis.

The relief zone can be made such that the bending stiffness is lowest in a plane which is spanned from the longitudinal axis of the shaft and the direction of an intervertebral force applied via the head in the implanted state, with the bending stiffness being largest in a plane spanned perpendicular thereto in an embodiment.

The shaft can have a rotationally asymmetrical cross-section in the region of the relief zone and can in particular have substantially an identical dimension to the directly adjacent upper and lower shaft regions in the plane of the largest bending stiffness.

It can be achieved by such an embodiment that the stiffness of the shaft with respect to torsion and with respect to tractive/compressive forces is impaired as little as possible. With a pedicle screw, the screw-in behavior is impaired as little as possible by this embodiment.

In the relief zone, the shaft can have at least one slot passing through the shaft and extending substantially in the longitudinal direction of the shaft. A region of the shaft slit in this manner can so-to-say be called a helix with an “infinitely large” helical pitch and in this respect represents a special case of a helical or double helical relief zone and can likewise be manufactured as described above by a wire erosion process without rotation of the screw.

The upper shaft region can have at least one recess extending substantially in the longitudinal direction at the surface. The bone can grow into recesses of this kind, whereby a security against rotation is provided.

The shaft can be made in one piece.

The relief zone can be made at least partly from an intermediate piece made of a material differing from the other shaft material.

The intermediate piece can be manufactured from a plastic material, in particular a fiber reinforced plastic material, in particular a polymer material.

Shaft regions adjacent to the relief zone can be connected to one another by a joint disposed in the relief zone.

The relief zone can be formed by a joint which connects two directly mutually adjacent shaft regions to one another.

In the embodiments which are provided with a shaft which is hollow at least regionally and with at least one helically extending interruption of the wall in this region, the manufacture of the interruption can take place in that edges are produced on the passing through of the thread peaks of a thread provided in this region, said edges being able to facilitate a screwing in of the pedicle screw.

The manufacture of a pedicle screw can take place by a wire erosion process in which, starting from an at least regionally hollow shaft, a wire extending through the shaft is guided relative to the pedicle screw such that two helically extending slot-like interruptions with a predetermined helix pitch are produced in the wall.

Deflection experiments on pedicle screws manufactured in this manner have shown that, starting from conventional pedicle screws in which, for instance a force of 150 N is required for a deflection of 1 mm, this force is reduced to 15 N when the helix pitch amounts to approximately 9 mm. It was further shown that it can be achieved by a reduction in the helix pitch that a force of 1 N can achieve a deflection of more than 1 mm. It was therefore shown that the bending stiffness can be shown as a function primarily of the helix pitch.

The shaft of the fastening element can have a circular cross-section. However, a circular cylindrical shape of this type is not absolutely necessary. It is rather possible for the shaft to have a cross-section differing from a circular shape, for example an elliptical or oval cross-section. The circumstance can hereby be taken into account that pedicles do not have a circular cylindrical shape, but an oval cross-section. A fastening element having a correspondingly shaped shaft can therefore support itself better at the cortical wall of the pedicle than a shaft having a circular cylindrical shape, whereby an improved anchorage in the pedicle can be generated.

The shaft can be made of a memory metal, for example of a memory metal on a NiTi base.

Intervertebral stabilization systems having fastening elements to be anchored in the vertebral bodies through the pedicle, such as in particular pedicle screws, are generally known. As already initially explained, it is generally possible to distinguish between rigid or angle-stable systems or systems with an angle-invariant coupling, on the one hand, and dynamic or elastic systems or systems with an angle-variant coupling, on the other hand. A dynamic intervertebral stabilization system is sold, for example, by the applicant under the product name “Dynesys” and is described, for example, in EP 669 109.

It is common to all stabilization systems of this type that their fastening systems, in particular pedicle screws, are anchored in at least two adjacent vertebrae and are connected to one another by a connection device. A plurality of possibilities exist for the design of this connection device. For instance, the connection device can, for example, include a rigid or a resilient rod by which at least two fastening elements or pedicle screws are rigidly connected to one another.

For the realization of a dynamic or resilient system, the connection device can include a band which can be pre-tensioned and which is surrounded in the implanted state by at least one compressible pressure member arranged between two adjacent pedicle screws (“Dynesys”).

The pedicle screws set forth here can now be generally provided in all intervertebral stabilization systems. Since intervertebral stabilization systems are generally known per se, these systems will not be looked at in any detail in the following.

The invention will be described in the following by way of example with reference to the enclosed drawing. There are shown:

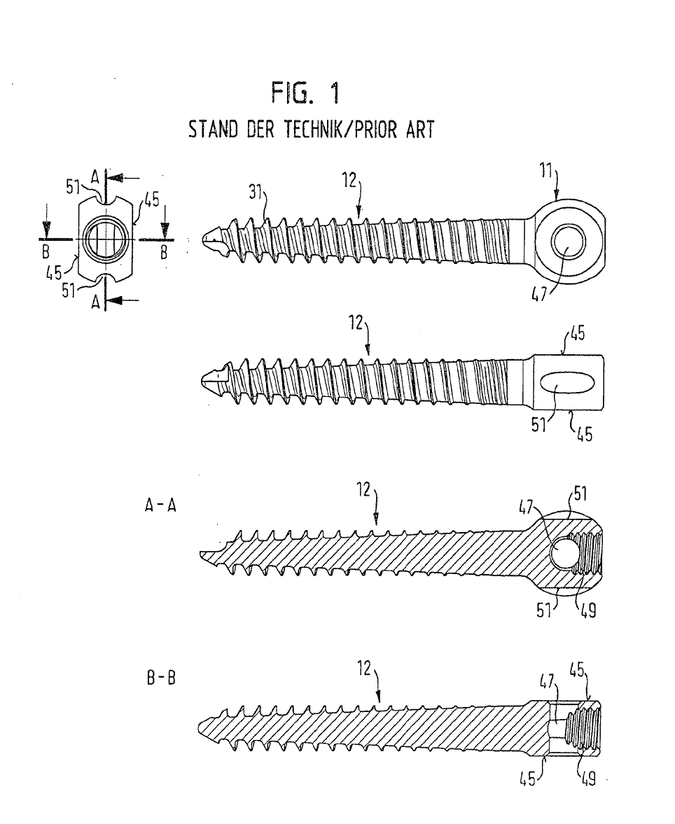

FIG. 1 a pedicle screw in accordance with the prior art;

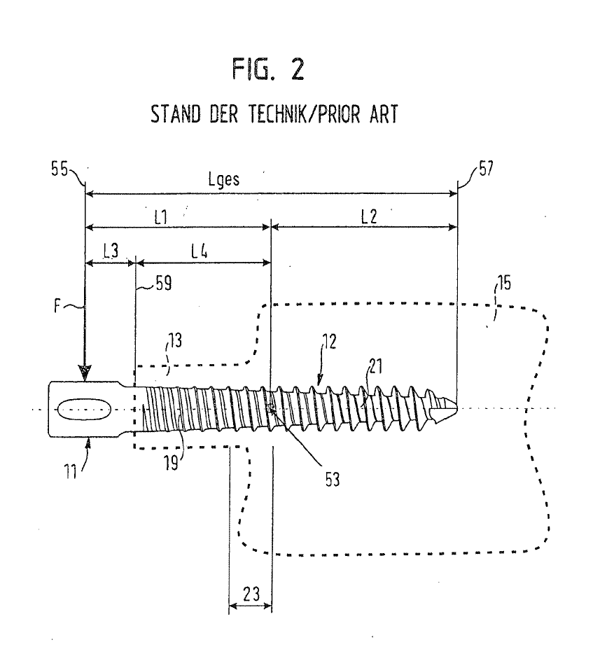

FIG. 2 the pedicle screw of FIG. 1 together with a vertebra for the explanation of specific dimensions;

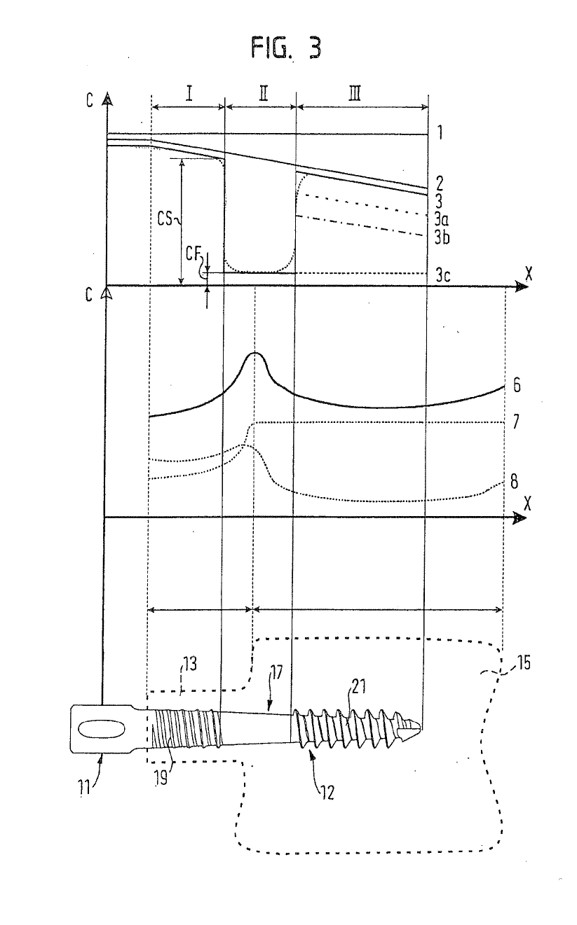

FIG. 3 a fastening element together with curves of different bending stiffness profiles;

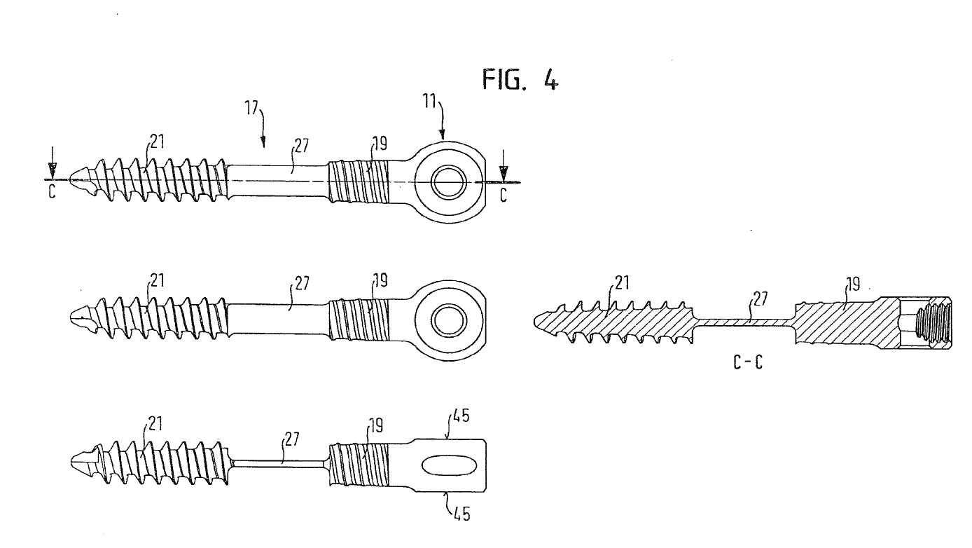

FIGS. 4-16 different embodiments of fastening elements; and

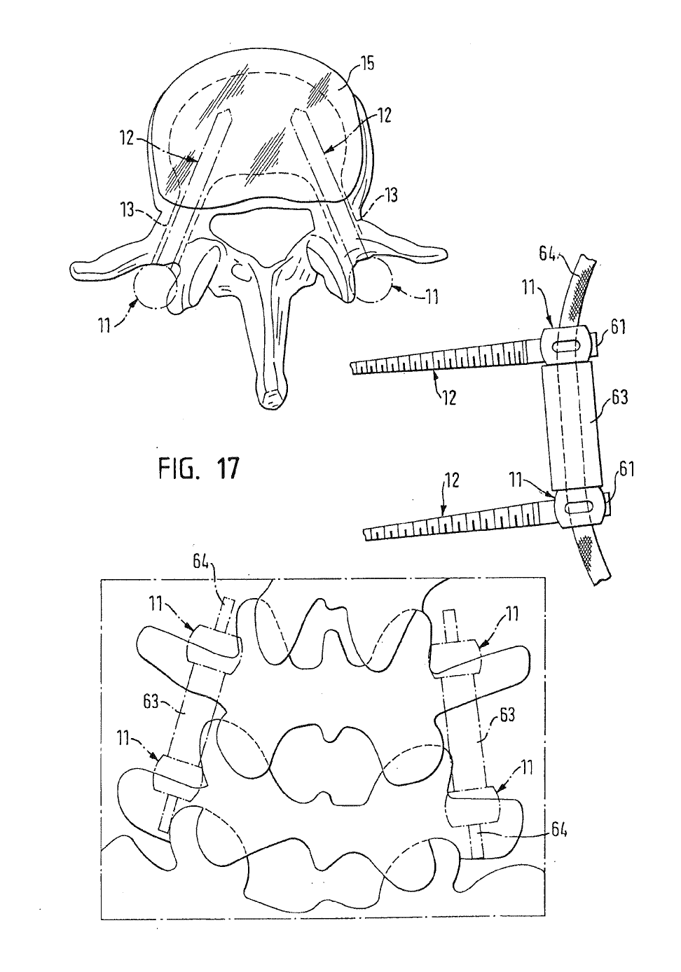

FIG. 17 a representation for the explanation of an intervertebral stabilization system.

The fastening elements described in the following are designed for a dynamic or resilient intervertebral stabilization system, for example for the Dynesys system of the applicant, such as has already been explained above (cf. also FIG. 17 with associated explanation at the end of the description). Fastening elements such as pedicle screws for intervertebral stabilization systems vary above all in the region of the head since the head serves for the coupling with the connection device, by which fastening elements or pedicle screws of adjacent vertebrae are connected to one another. The heads of the fastening elements described in the following are designed for the Dynesys system of the applicant. The design of the shaft of the individual fastening elements can basically be combined with heads of any desired design and is therefore generally usable for any desired intervertebral stabilization systems.

FIGS. 1 and 2 show a pedicle screw designed for the Dynesys system of the applicant and belonging to the prior art. The shaft 12 having a circular cross-section is provided with a thread 31 and is conical. The head 11 is provided on mutually oppositely disposed sides with holding depressions 51 which serve for the holding of the screw by means of a correspondingly designed instrument during the implanting. The head 11 is flattened on the other two sides disposed opposite one another. The planar support surfaces 45 hereby formed serve for the support of cylindrical, compressible pressure members of the stabilization system (not shown). A band of the stabilization system which can be pre-tensioned is led through this resilient pressure member and through a passage 47 made in the head 11 and is fixed to the head 11 by means of a fixing screw (not shown) which is screwed into a thread 49 formed in the head 11. In the implanted state, the band is pre-tensioned, whereas the resilient pressure member arranged between two screw heads 11 and supported at the support surfaces 45 is compressed. A dynamic stabilization system is hereby realized overall which reacts resiliently both to pressure strain and to tension strain perpendicular to the screw axis.

FIG. 2 schematically shows a known pedicle screw of this kind which is implanted into a vertebra and in this process extends with its shaft 12 through the pedicle 13 up to and into the vertebral body 15. As initially explained, rigid pedicle screws are prone to tilting or rotation about a so-called toggle point or pivot point 53, which—as investigations have shown—lies in the transition region 23 between the pedicle 13 and the vertebral body 15, under the effect of an intervertebral force F applied via the head 11.

While taking account of the characteristic regions of the vertebra and of the position of the toggle point 53 determined by extensive tests, the minimum dimensions, maximum dimensions and average dimensions (all figures in mm) recited in the following table apply both to the known pedicle screws in accordance with FIG. 2 and to the fastening elements described in the following. The following parameters are listed in the table, with “band” meaning the previously mentioned band of the stabilization system which is capable of being pre-tensioned and is guided through the passages 47 formed in the heads 11 and located between the heads 11:

Lges: axial length of the band axis 55 up to the screw tip 57
L1: axial length of the band axis 55 up to the toggle point 53
L2: axial length from the toggle point 53 up to the screw tip 57
L3: axial length from the band axis 55 up to the bone entry 59
L4: axial length from the bone entry 59 up to the toggle point 53
D1: diameter at the screw tip 57
D2: diameter at the toggle point 53
D3: diameter at the bone entry 59

| [mm] | Minimum | Maximum | Average |
|---|---|---|---|
| Lges | 40 | 60 | 50 |
| L1 | 8 | 40 | 30 |
| L2 | 0 | 40 | 20 |
| L3 | 6 | 15 | 10 |
| L4 | 10 | 30 | 20 |
| D1 | 2.8 | 5.5 | 3.4 |
| D2 | 4 | 6.75 | 4.9 |
| D3 | 5.2 | 8 | 6.4 |

The dimensions to be used in a specific case are dependent on, among other things, the technique used in the positioning of the pedicle screws.

The bending stiffness C measured in the direction of the longitudinal axis of the implanted fastening element can be defined as the product of the modulus of elasticity and the surface torque of the second order (surface inertial torque). The bending stiffness profile of the fastening element is particularly well suited to show the difference between conventional rigid pedicle screws, on the one hand, and the flexible fastening elements set forth here on the other hand. The bending stiffness of the fastening element can be influenced by the selection of the material for the shaft and by the shaft geometry. In the first case, the modulus of elasticity varies, whereas in the second case the surface torque is changed.

FIG. 3 shows, in the bottom third, a fastening element which is provided in the form of a pedicle screw and has a head 11 and a shaft 12. The shaft 12 is provided with a relief zone 17 (zone II). Details of this relief zone 17 will not be looked at in more detail here. Specific embodiments are explained in the following. FIG. 3 serves for the explanation of general relationships.

In the upper third of FIG. 3, bending stiffness profiles are shown for differently made fastening elements. The middle third of FIG. 3 shows the bending stiffness profile of the bone structure into which the pedicle screw is implanted, i.e. the bone structure of pedicle 13 and vertebral body 15. The bending stiffness profiles shown have been measured along the central axis of the shaft 12 of the fastening element which coincides with the central axis of the pedicle 13 at least approximately.

The representation of FIG. 3 is divided into three zones along the shaft 12. In the following, the shaft region between the head 11 and the start of the relief zone 17 is also termed the upper shaft region 19 (zone I), whereas the region of the shaft 12 behind the relief zone 17—considered from the head 11—is also termed the lower shaft region 21 (zone III).

Curve 1 shows the bending stiffness profile of a conventional rigid pedicle screw assumed to be ideally cylindrical without a relief zone. The bending stiffness C is constant over the total shaft length.

Curve 2 shows the bending stiffness profile of a conical pedicle screw tapering in the direction of the free shaft end or of a pedicle screw with a conical core. The bending stiffness reduces constantly in this case.

The curves 3, 3a, 3b and 3c show bending stiffness profiles of fastening elements which are each provided with a relief zone or a flexible region 17. The relief zone 17 starts before the transition between the pedicle 13 and the vertebral body 15 and ends behind this transition inside the vertebral body 15. The bending stiffness profile has a respective cup-like extent with steep walls, i.e. the reduction or increase in the ending stiffness takes place abruptly. The transitions can be more or less pronouncedly step-like in shape. An example is shown by the dotted curve in which the transitions are rounded with respect to the extent shown with a solid line.

The curves 3a and 3b show bending stiffness profiles which each correspond to the extent of the curve 3, with the difference that the lower shaft region 21 (zone III) is provided with a significantly reduced stiffness which, however, still lies considerably above that in the relief zone 17 (zone II).

In the example of curve 3c, the bending stiffness in the lower shaft region 21 corresponds to that in the relief zone 17. In other words: in this example, the relief zone 17 extends up to the tip at the free end of the fastening element.

The upper third of FIG. 3 furthermore shows that the bending stiffness CF in the relief zone 17 (zone II) only amounts to approximately a tenth of the corresponding value CS in the upper shaft region 19 (zone I).

In the middle third of FIG. 3, curve 7 shows the axial extent of the modulus of elasticity of the bone structure made up of the pedicle 13 and the vertebral body 15. The corresponding extent of the surface torque of the second order of the bone is shown in curve 8. The sum of the two curves 7 and 8 is shown as the curve 6 with a solid line which, in this simplified model, represents the profile of the bending stiffness C of the bone structure made up of the pedicle 13 and the vertebral body 15.

It results from this that the bending stiffness of the vertebra is the largest in the region of the transition between the pedicle 13 and the vertebral body 15. The comparison with the upper third of FIG. 3 shows that the relief zone 17 can be positioned in the shaft 12 such that, with an implanted fastening element corresponding to the lower third of FIG. 3, the transition between the pedicle 13 and the vertebral body 15 and thus the region of maximum bending stiffness of the vertebra is disposed inside the relief zone 17 of the shaft 12 and in particular centrally—considered in the axial direction—in the relief zone 17.

Possible lengths for the zones I, II and II shown in FIG. 3 of the shaft 12 of the fastening element are as follows:

The axial length of zone I and thus approximately the spacing from the lower side of the head 11 up to the start of the relief zone 17 can lie between 8 mm and 35 mm. The smallest length can be used for the vertebra L5 if the screw is screwed in so far that the screw head 11 contacts the bone. The largest length is used when a so-called posterior medial screw setting technique is used, with the system being positioned to posterior of the facet joints.

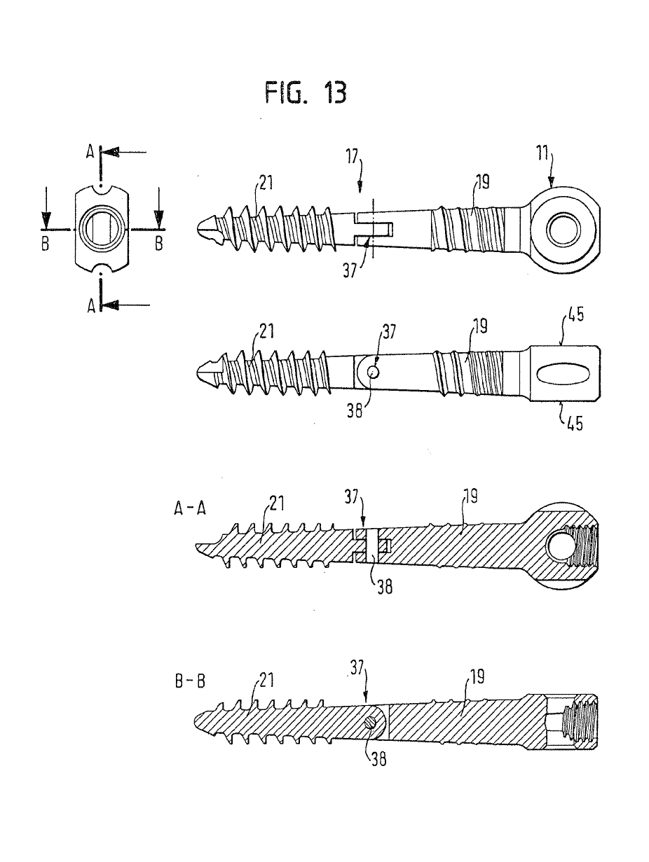

A range from 0 mm to 35 mm can be provided for the length of zone II, i.e. of the relief zone or of the flexible transition zone 17. The smallest length can be present when the relief zone 17 is made as a joint (cf. the following embodiment in FIG. 13). In this case, the relief zone 17 is practically formed by the axis of rotation of the joint. The largest length can be present when the relief zone 17 extends up to the tip of the fastening element.

A range from 0 mm up to 35 mm can be provided for the length of the lower shaft region 21. The smallest length is present when no lower shaft region 21 in the sense of FIG. 3 is provided, but rather the relief zone 17 extends up to the tip of the relief element. The largest length can be present when the free end of the fastening element contacts the anterior cortex or even penetrates it (so-called bicortical screw setting technology).

In the embodiment of a pedicle screw in FIG. 4, the relief zone 17 is formed by material removal at the shaft. Consequently, the relief zone 17 here is a shaft region 27 with a reduced cross-sectional area with respect to the adjoining shaft regions 19, 21. The cross-section in the relief zone 17 is not circular, but the shaft region 27 is rather made in the shape of a leaf spring and is provided in the form of a thin strip. The width of the strip corresponds to the original shaft diameter, whereas the strip thickness is substantially smaller than the original shaft diameter.

In this example, the bending stiffness of the shaft is thus rotationally asymmetrical with respect to its longitudinal axis. The orientation of the relief zone 17 is selected such that the plane defined by the strip 27 extends parallel to the planar support surfaces 45 at the screw head 11. The relief zone 17 is therefore only fully effective in one plane, with this plane being spanned by the longitudinal axis of the shaft and a normal of the planar support surfaces 45.

A first rough dimensioning of a screw in accordance with FIG. 4 has resulted in the following: the starting point was formed by a conventional pedicle screw (without a relief zone) of the Dynesys system of the applicant whose material (a titanium alloy) has a modulus of elasticity of E=105,000 N/mm². While observing the criterion of fatigue strength (dynamic load capability or alternate bending strength under a maximum deformation of 10°), the following average dimensions resulted for the relief zone 17:

| | | |
|---|---|---|
| Length: | 15 | mm |
| Width: | 6.5 | mm |
| Height: | 1 | mm |

Rough calculations showed that only approximately 10% of the maximum permitted surface pressure can be caused in the vertebral body with a screw of this type. In other words, in simplified terms: a screw of this type is less stable than the vertebral body would allow.

This calculation example—which therefore does not result in an optimum fastening element, but is nevertheless suitable for the explanation of the basic principle of the pedicle screw set forth here—therefore shows that the relief zone determines the load capability of the fastening element, on the one hand, and the load of the vertebral body, on the other hand: On the one hand, the bending stiffness in the relief zone should only be reduced so far that the load capability of the vertebral body is just not exceeded, for the greater the bending stiffness of the relief zone is made, the greater forces or torques an be transmitted into the vertebral bodies, which is generally desired, since the free end region of the fastening element should carry along the maximum, but actually without overloading the vertebral body (and so causing loosening). While observing the maximum load capability of the vertebral body, a highest possible bending stiffness in the relief zone is aimed for, on the one hand. On the other hand, as the bending stiffness of the relief zone increases, the load capability of the fastening element is reached earlier.

The fastening element is consequently configured such that an optimum “compromise” is adopted between these two basically opposite optimization criteria.

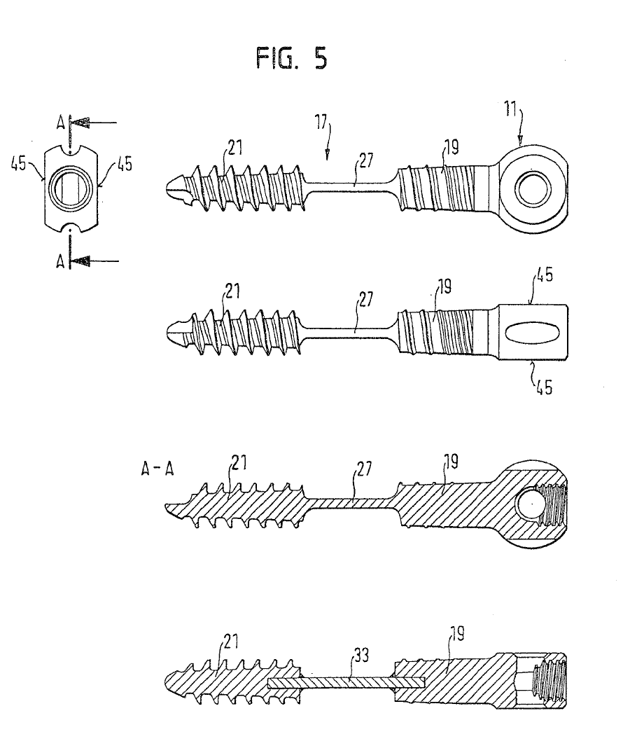

In the example of FIG. 5, contrary to the design shown in FIG. 4, the shaft region 27 forming the relief zone 17 is provided with a circular cross-section. The bending stiffness profile of the screw is thus here rotationally symmetrical with respect to its longitudinal axis.

In the lower representation in FIG. 5, a variant is shown in which the shaft region forming the relief zone 17 is not made in one piece, but is rather formed by a separate intermediate piece 33 which is anchored in the upper and lower shaft regions 19, 21. The intermediate piece 33 can be a rod made of a material of particularly low bending stiffness (“superelastic” material). It is in particular a material which can be deformed up to 10% or more without permanent deformation, which is not achieved by the usual implant steels or by titanium. A memory metal on the basis of NiTi (e.g. nitinol) can in particular be considered for the intermediate piece 33.

It is also possible to provide an element made in the form of wire rope as the intermediate piece 33.

Whereas the cross-sectional surface is reduced and thus the surface torque of the shaft is lowered on a weakening of the shaft by material removal, the modulus of elasticity of the shaft can be changed directly by a change of the material in the relief zone 17.

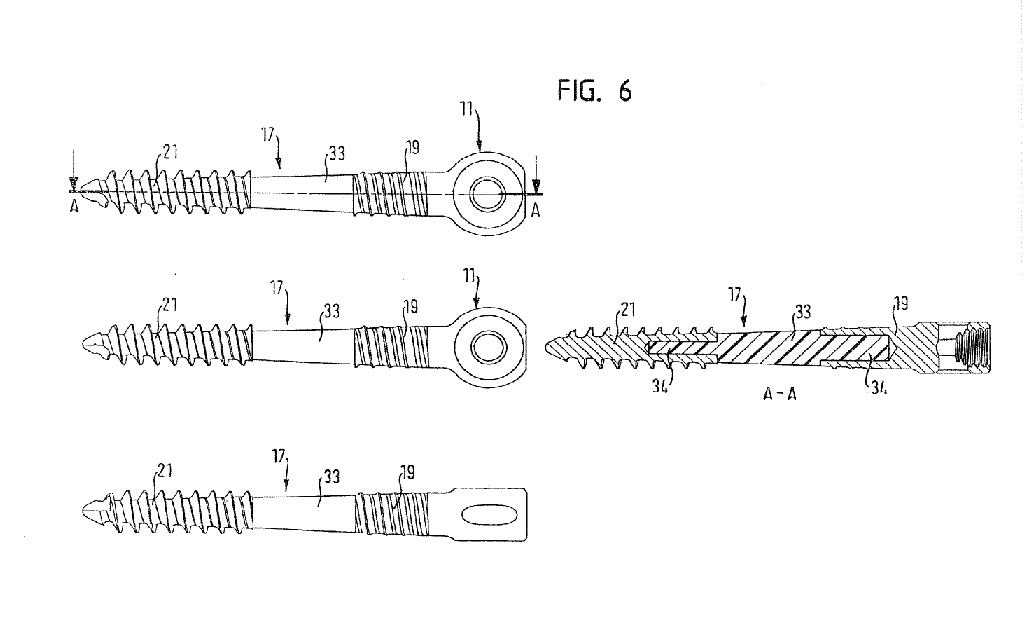

A further example for such a reduction of the modulus of elasticity by material change is shown in FIG. 6. Unlike in the example in the lower representation in FIG. 5, no reduction in cross-section takes place here, but only a change of material.

A biocompatible titanium alloy or steel alloy is in particular used as the material for the pedicle screw set forth here and thus for the lower and upper shaft regions 19, 21 and for the head screw 11. A material is used for a separate intermediate piece 33 forming the relief zone 17 here which has a much lower modulus of elasticity than the shaft material and so provides a corresponding flexibility or durability of the shaft. A possible material for the intermediate piece 33 is plastic, in particular an elastomer. The plastic can be fiber reinforced. The bending stiffness of the shaft can generally be set as desired by the material selection and thus by the pre-setting of the modulus of elasticity.

As the cross-sectional representation at the right in FIG. 6 shows, the intermediate piece 33 made conically in accordance with the extent of the shaft is provided with anchorage prolongations 34 which extend at both sides in the axial direction and via which the intermediate piece 33 is fastened to the adjacent shaft regions 19, 21.

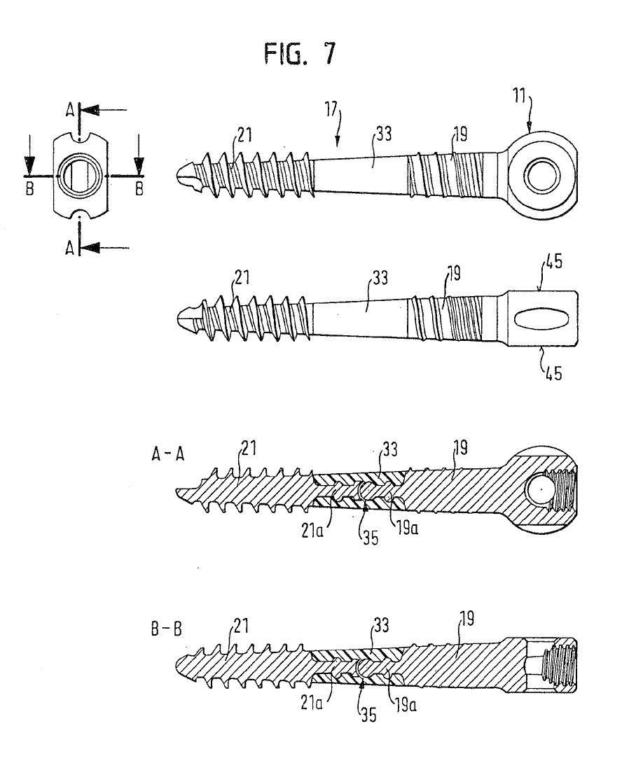

FIG. 7 shows a variant in which an intermediate piece 33 manufactured, for example, from plastic material is likewise provided in order to form the relief zone 17. The anchorage at the adjoining shaft regions 19, 21 takes place by axial projections 19a, 21a which are formed in one piece at these shaft sections 19, 21 and which are provided with radial widened sections to improve the shape matching.

One special feature of this variant consists of the prolongations 19a, 21a cooperating in the manner of a joint approximately at the center of the relief zone 17 inside the intermediate piece 33; in the example shown here in accordance with the ball and cup principle, with a hinge arrangement, for example, alternatively being able to be provided. The upper shaft section 19 and the lower shaft section 21 are therefore connected to one another in this example by a joint 35 which is surrounded by the elastic material of the intermediate piece 33, whereby the corresponding restoration forces are provided in the case of a deflection of the shaft. The joint region forming the relief zone 17 can in particular have the material forming the intermediate piece 33 injection molded around it.

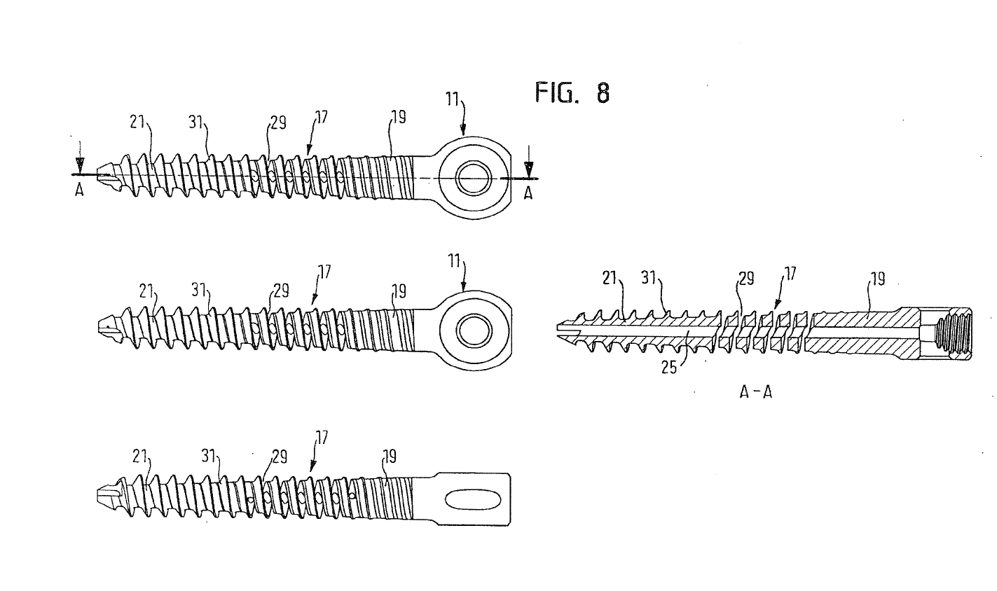

FIGS. 8, 9 and 10 show embodiments in which the surface torque is in turn reduced and the shaft material is simultaneously ideally used.

In the example of FIG. 8, the shaft is provided with a central bore 25. This is of advantage from a technical production aspect and moreover permits the pedicle screws to be implanted with the aid of a Kirschner wire, which is in particular advantageous with percutaneous implanting.

Unlike in the previously explained examples, the thread 31 of the screw is made in uninterrupted form here. In the region of the relief zone 17, the wall of the shaft is broken open in the thread valley. The shaft is hereby made in the manner of a helical spring or of a corkscrew in the region of the relief zone 17. A relief zone of this kind can be produced without problem, for example, by wire erosion or by a powered side milling cutter.

The peripheral slot in the thread valley in the relief zone can have a much smaller slot width than shown in FIG. 8. The slot width can in particular be selected to be so small that, on a specific bending of the shaft, the windings in the relief zone abut or lie on one another such that pressure forces can hereby be transmitted.

It is furthermore possible for the slot formed only in the relief zone 17 in accordance with FIG. 8 to extend up to the screw tip, i.e. for the whole remaining shaft adjoining the upper shaft region 19 to be made in the manner of a helical screw or of a corkscrew.

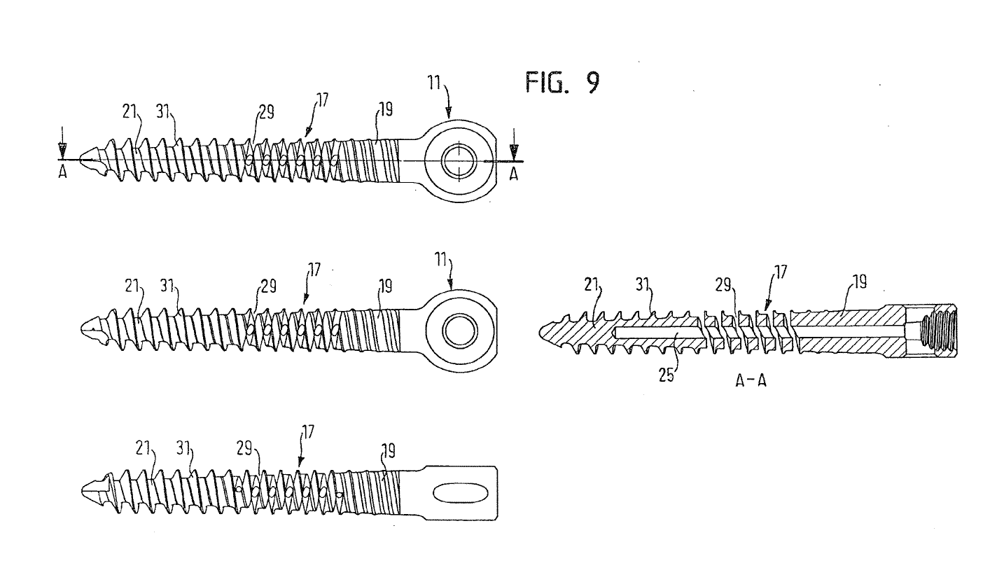

In a further embodiment, the slots can be filled with a bioabsorbable material which reduces or cancels the spring effect. The intraoperative stability of the shaft can hereby be increased. Depending on the design of the filler material, the absorption takes place comparatively fast or within some days. The flexibility of the screw pre-determined by the shaft geometry is then fully effective subsequent to the absorption.

Whereas the slot formed in the shaft wall is made in the same sense with the thread 31 in the example of FIG. 8, in accordance with the example of FIG. 9, the slot can also be made in the opposite sense to the thread 31. When the screw is screwed in, the shaft diameter or core diameter is restricted in the region of the relief zone 17 by the friction forces which become effective in this process. The springing back of the shaft after the removal of the screw-in force effects a further compression of the surrounding bone material, which can substantially improve the primary stability of the screw.

The provision of an uninterrupted central bore, as in the example of FIG. 8, is not absolutely necessary. As the example of FIG. 9 shows, a central bore 25 can also be provided which only extends partly through the shaft. In this example, the bore 25 ends in the region of the lower shaft section 21.

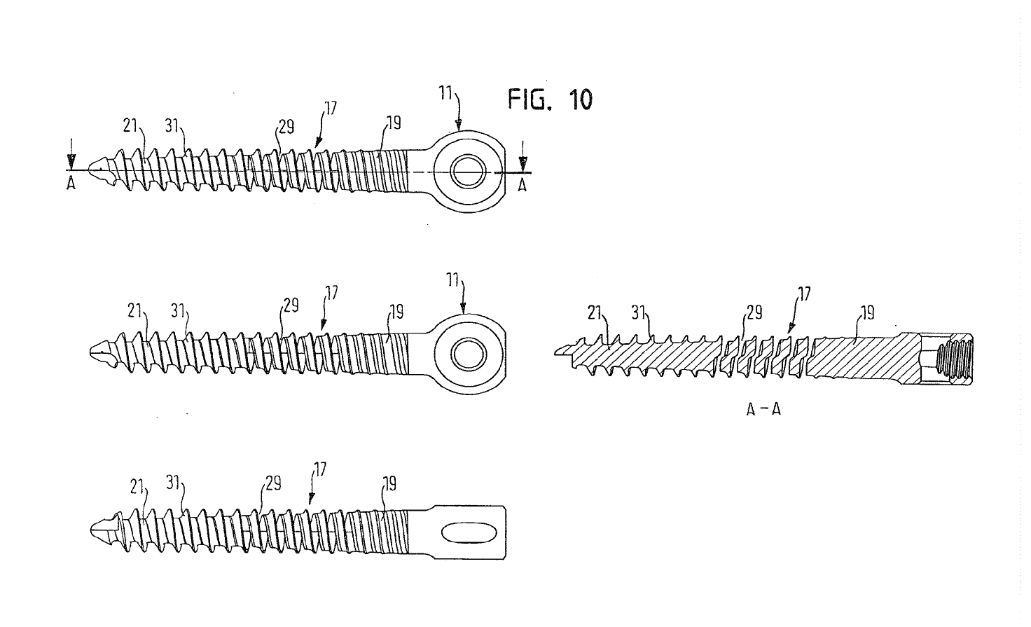

As the example of FIG. 10 shows, it is also possible with shafts without a central bore to provide spiral or helical peripheral recesses in order to form the relief zone 17. Structures forming a helix can also be manufactured in this case by a correspondingly selected plunge-cut depth which make a central bore superfluous to this extent. The orientation of the recesses or slots can be selected in accordance with the examples of FIGS. 8 and 9 either to be in the same sense or in the opposite sense with respect to the screw thread 31.

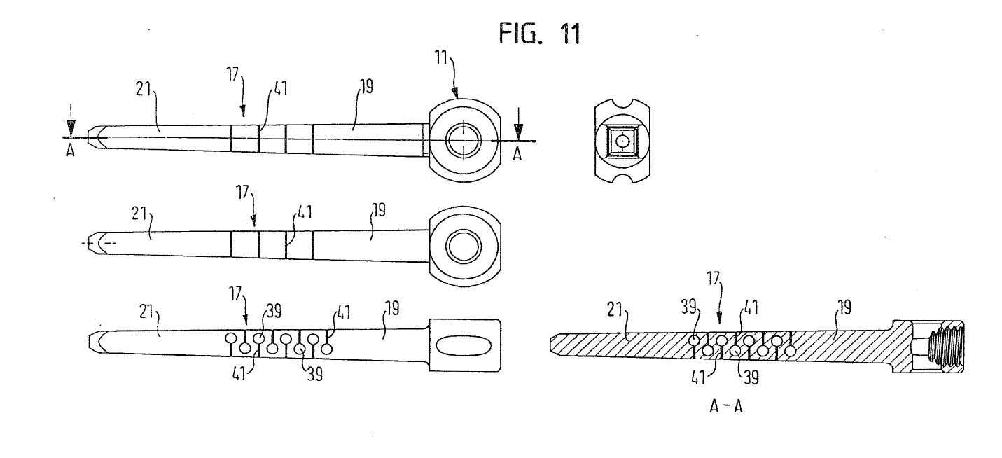

The embodiment of FIG. 11 includes a plurality of special features which will be explained in the following, with such a combination of features not being absolutely necessary, but the individual aspects rather also being able to be realized independently of one another in connection with other embodiments.

The fastening element of FIG. 11 is not a screw, but a pin-like fastening element which is not screwed in, but rather hammered into the vertebra.

In order to form the relief zone 17, the shaft is provided with slots 41 which are arranged offset, have a low width and each open into an uninterrupted bore 39. The blow impulses required for the hammering in can be transmitted without problem in the axial direction due to the low slot width.

The cross-section of the shaft can differ from a circular shape and can in particular be made in oval or elliptical shape in order hereby to achieve a better match to the natural pedicle shape, as was already initially explained.

Furthermore, the shaft of the fastening pin to be hammered in can be provided with a thread present with a start in order to facilitate the explanting of the fastening element in the case of a reoperation.

It must be explicitly noted that screws provided with a slot-like or groove-like, helical or spiral peripheral recess, such as were explained above, can also have a shaft with a cross-section differing from a circular shape, i.e. non-rotationally symmetrical shaft geometries are not limited to hammer-in pins in accordance with FIG. 11.

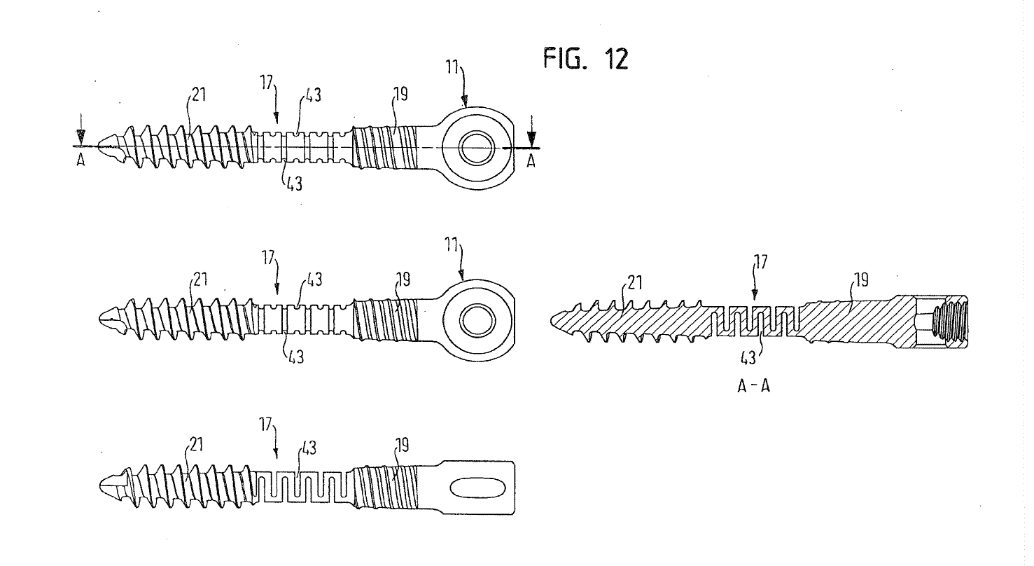

FIG. 12 shows an example with relief slots or relief notches 43 which are arranged in offset form and whose width is in each case larger than that of the slots 41 in the example of FIG. 11. Relief notches of this type can be provided both with shafts having circular cross-sections and with shafts having cross-sections differing from a circular shape.

It is furthermore possible to reduce the bending stiffness of the shaft to zero. For this purpose, for example in accordance with the embodiment of FIG. 13, a rotary joint 37 can be provided between the upper shaft section 19 and the lower shaft section 21. A joint pin 38 of the joint 37 defining the axis of rotation extends parallel to the planar support surfaces 45 of the screw head 11. The position of the joint 37 along the shaft axis can be disposed such that the axis of rotation of the joint 37 lies in the region of the aforementioned toggle point (FIG. 3) when the screw is implanted and in particular runs through the toggle point.

In the case of a rotational joint 37 of this kind, no relief zone is present in the sense of the embodiments described above which extends over a significant axial length of the shaft. In the example of FIG. 13, it is rather the joint 37 itself that forms the relief zone 17.

It is furthermore possible to design the relief zone such that its bending stiffness increases over time. For example, a plastic material can be used for the relief zone whose hardness increases over time, whereby the force transmission or torque transmission onto the free end region of the fastening element increases accordingly. The circumstance can hereby be taken into account that, after a specific time following the operation, it can be assumed that the fastening material has grown well into the bone material. The flexibility of the shaft, which is still relatively high initially, in particular therefore serves the purpose in this process of avoiding excessive strains shortly after the operation.

Alternatively—as already mentioned above in connection with the example of FIG. 8—the shaft can be made such that the bending stiffness decreases over time in the relief zone. For this purpose, absorbable components can, for example, be provided which have a stiffening effect on implanting and which are absorbed over time, whereby the flexibility of the shaft is gradually increased in the region of the relief zone.

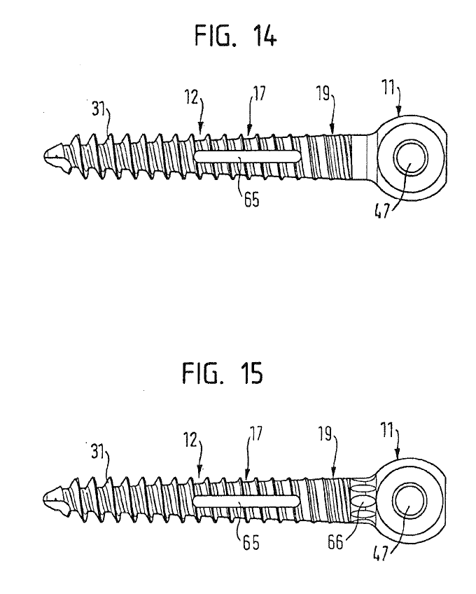

FIG. 14 shows a pedicle screw whose shaft 12 has a slot 65 passing through the shaft 12 and extending in the longitudinal direction of the shaft 12 to form the relief zone 17.

In FIG. 15, it is shown for the example of the pedicle screw of FIG. 14 that the upper shaft region 19 can have recesses 66 extending in the longitudinal direction at the surface, with the bone being able to grow into these recesses, whereby a security against rotation is provided.

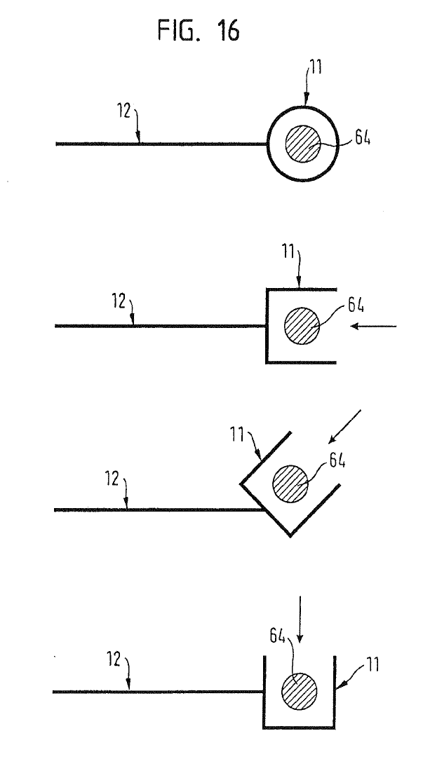

In FIG. 16, a plurality of possible embodiments of the head 11 of a fastening element of the kind set forth here are shown—purely schematically—which differ in how a connection element 64 of an intervertebral element 64 of an intervertebral stabilization system—for example a band 64 in the Dynesys system of the applicant (see also the following description of FIG. 17)—can be received in or at the head 11.

Whereas with the upper embodiment, the head 11 is made as a ring or eye through which the connection element 64 is guided, in the other embodiments the head 11 is made in the manner of a “U” which stands upright, is inclined or lies on its side—with respect to the longitudinal axis of the fastening element—such that the connection element 64 does not have to be “threaded through” an opening, but can be inserted (in the direction of the arrow), and indeed either from above (so-called top loading principle) or from the side (so-called “side loading principle”). All the embodiments of the pedicle screw set forth here described in the above, in particular with reference to FIGS. 3 to 15, can generally be provided with any of the head variants shown in FIG. 16.

The “top loading” principle is described, for example, in EP 528 706.

It is common in the prior art to fix the connection element 64 by a screw in particular screwed in the direction of insertion into a thread arranged at the head 11.

FIG. 17 shows, in a plurality of representations, an example for an intervertebral stabilization system which can include fastening elements set forth here, in particular in the form of pedicle screws. The dynamic system shown is an elastic support system (Dynesys system of the applicant) such as was mentioned a plurality of times above.

Adjacent vertebrae are connected to one another by part systems of the same construction. Two pedicle screws are screwed into each vertebra, each have a shaft 12 and a head 11 and each extend through a pedicle 13 into the vertebral body 15. In both part systems, a compressible pressure member or support member 63 is respectively arranged between two screw heads 11. A band 64 pre-tensioned in the implanted state and fixed in the heads 11 by fixing screws 61 extends through the pressure member 63 and the heads 11. Pulling forces are absorbed elastically by the belt 64 and pressure forces with the pressure member. The pedicle screws shown can be fastening elements of the kind set forth here.

### REFERENCE NUMERAL LIST

11 head
12 shaft
13 pedicle
15 vertebral body
17 relief zone
19 upper shaft region
19a joint prolongation
21 lower shaft region
21a joint prolongation
23 transition region
25 central bore
27 shaft region with reduced cross-sectional area
29 spiral-shaped recess, slot
31 thread
33 intermediate piece
34 anchorage prolongation
35 joint in the relief zone
37 joint as the relief zone
38 joint pin
39 transverse bore
41 slot
43 slot-like, groove-like or notch-like recess
45 planar support surface
47 passage
49 thread opening
51 holding depression
53 pivot point, toggle point
55 band axis
57 screw tip
59 bone entry
61 fixing screw
63 pressure member or support member
64 band
65 slot
66 recess
F intervertebral force

1-40. (canceled)

41. A pedicle screw for use in an intervertebral stabilization system having an intervertebral stabilization element, the pedicle screw comprising:
a head adapted for coupling to the intervertebral stabilization element of the intervertebral stabilization system;
a shaft adapted to be inserted at least partially into a vertebra through a pedicle area of the vertebra;
an upper shaft region of the shaft adjacent to the head and having an upper bending stiffness;
a lower shaft region of the shaft spaced from the head and having a lower bending stiffness; and
a relief zone located on the shaft and intermediate the upper and lower shaft regions, the relief zone having a reduced bending stiffness with respect to the upper bending stiffness and the lower bending stiffness and being located on the shaft to be embedded in the vertebra.

42. The pedicle screw of claim 41 wherein the head is rigidly coupled to the upper shaft region at least in a bending direction of the shaft.

43. The pedicle screw of claim 41 wherein a first gradient of the bending stiffness at a transition area from the upper shaft region to the relief zone is at least twice as large as a second bending stiffness gradient occurring in the upper shaft region, and a third gradient of the bending stiffness at a transition area from the relief zone to the lower shaft region is at least twice as large as a fourth gradient of the bending stiffness occurring in the lower shaft region.

44. The pedicle screw of claim 41 wherein the shaft is dimensioned such that the relief zone is adapted to be disposed in the spongiosa and the upper shaft region in the cortex.

45. The pedicle screw of claim 41 wherein an axial bending stiffness profile of the shaft generally corresponds to a bending stiffness profile of the pedicle area.

46. The pedicle screw of claim 41 wherein the relief zone is disposed in a central region of the longitudinal extent of the shaft.

47. The pedicle screw of claim 41 wherein the upper shaft region and the relief zone are dimensioned such that when the pedicle screw is implanted in the vertebra, the relief zone is in the region of the transition between the pedicle area and the vertebral body and extends axially beyond the transition region on both sides of the vertebral body, with a section of the relief zone being disposed behind the transition region when viewed from the head having a larger axial length than a section of the relief zone disposed in front of the transition region.

48. The pedicle screw of claim 41 further comprising:
a thread on the shaft being interrupted by the relief zone.

49. The pedicle screw of claim 41 wherein the shaft includes a central bore.

50. The pedicle screw of claim 41 wherein the relief zone includes a cross-section attenuation relative to at least one of the upper shaft region and the lower shaft region.

51. The pedicle screw of claim 41 wherein the relief zone is formed by an elongated shaft region having a reduced cross-sectional area relative to at least one of the upper shaft region and the lower shaft region.

52. The pedicle screw of claim 41 further comprising:
at least one helically shaped recess in the relief zone.

53. The pedicle screw of claim 52 wherein a pitch of the helical shape varies over the longitudinal extent of the shaft.

54. The pedicle screw of claim 41 further comprising:
two slot-like openings having a helical periphery in the relief zone.

55. The pedicle screw of claim 54 wherein the shaft forms a double helix in the relief zone.

56. The pedicle screw of claim 41 further comprising:
transverse bores extending perpendicular to the longitudinal axis of the relief zone.

57. The pedicle screw of claim 56 further comprising:
at least one slit leading from an outer surface of the shaft to the transverse bores and whose width is smaller than a diameter of the transverse bores.

58. The pedicle screw of claim 41 further comprising:
a plurality of offset recesses arranged serially in the axial direction of the shaft and a depth of each recess being larger than half the diameter of the shaft measured in the region of the respective recess.

59. The pedicle screw of claim 41 wherein the bending stiffness profile of the shaft is rotationally asymmetrical with respect to its longitudinal axis.

60. The pedicle screw of claim 41 wherein the relief zone is configured such that the bending stiffness is lowest in a plane which is spanned from the longitudinal axis of the shaft and the direction of an intervertebral force applied via the head in the implanted state, with the bending stiffness being largest in a plane spanned perpendicular thereto.

61. The pedicle screw of claim 59 wherein the relief zone has a rotationally asymmetrical cross-section and substantially identical dimension in the plane of the largest bending stiffness to the directly adjacent upper and lower shaft regions.

62. The pedicle screw of claim 41 wherein the relief zone has at least one slot passing through the shaft and extending substantially in the longitudinal direction of the shaft.

63. The pedicle screw of claim 41 wherein the shaft is made in one piece.

64. The pedicle screw of claim 41 wherein the relief zone is made at least partly from an intermediate piece which is formed from a material differing from a material of a remainder of the shaft.

65. An intervertebral stabilization system comprising:
a plurality of pedicle screws; and
an intervertebral stabilization element coupling at least two adjacent pedicle screws anchored to adjacent vertebra;
wherein each of the pedicle screws further comprises
(a) a head coupled to the intervertebral stabilization element;
(b) a shaft inserted at least partially into the vertebra through a pedicle area of the vertebra;
(c) an upper shaft region of the shaft adjacent to the head and having an upper bending stiffness;
(d) a lower shaft region of the shaft spaced from the head and having a lower bending stiffness; and
(e) a relief zone located on the shaft and intermediate the upper and lower shaft regions, the relief zone having a reduced bending stiffness with respect to the upper bending stiffness and the lower bending stiffness and being located on the shaft to be embedded in the vertebra.

66. An intervertebral stabilization system according to claim 65 wherein the intervertebral stabilization element includes an elastic support system having a pre-tensionable band surrounded by at least one compressible pressure member arranged between the two adjacent pedicle screws.
